(19) United States
(12) Patent Application Publication
(10) Pub. No.: US 2026/0260706 A1
(43) Pub. Date: Sep. 3, 2026

(54) PREDICTIVE CONTROL METHOD, INFORMATION PROCESSING DEVICE, AND NON-TRANSITORY COMPUTER-READABLE RECORDING MEDIUM

(71) Applicant: Fujitsu Limited, Kawasaki-shi (JP)

(72) Inventors: Hiyori YOSHIKAWA, Kawasaki (JP); Fuyuka YAMADA, Kawasaki (JP); Mutsuyo WADA, Funabashi (JP); Kento OHGA, Yokohama (JP); Takashi KATOH, Kawasaki (JP); Akira NAKAGAWA, Sagamihara (JP)

(73) Assignee: Fujitsu Limited, Kawasaki-shi (JP)

(21) Appl. No.: 19/549,087

(22) Filed: Feb. 25, 2026

(30) Foreign Application Priority Data
Feb. 28, 2025 (JP) 2025-032449

(51) Int. Cl. G16B 40/00; G05B 13/02; G05B 13/04; G06T 15/08; G16B 15/00

(52) U.S. Cl. CPC G16B 40/00; G05B 13/0265; G05B 13/048; G06T 15/08; G16B 15/00

(57) ABSTRACT

A predictive control method includes acquiring first information that corresponds to a predicted structure predicted by a structure prediction model based on machine learning, and second information that is a fitting target of the first information, generating intermediate information between the first information and the second information based on the acquired first information and the acquired second information, and updating an intermediate feature amount of the structure prediction model based on the generated intermediate information.

### CROSS-REFERENCE TO RELATED APPLICATION

This application is based upon and claims the benefit of priority of the prior Japanese Patent Application No. 2025-032449, filed on Feb. 28, 2025, the entire contents of which are incorporated herein by reference.

### FIELD

The embodiments discussed herein are related to a predictive control method, an information processing device, and a computer-readable recording medium.

### BACKGROUND

A technique of predicting an atomic structure from an amino acid sequence is disclosed (refer to John Jumper, et. al “Highly accurate protein structure prediction with AlphaFold”, (online), (Searched on Feb. 6, 2025), the Internet <www.nature.com/articles/s41586-021-03819-2>). For example, a machine learning model (hereinafter, appropriately referred to as “structure prediction model”) such as AlphaFold2 or OpenFold outputs one typical atomic structure among atomic structures of protein for an input amino acid sequence.

FIG. 1 is a reference diagram illustrating a structure prediction model of AlphaFold2. As illustrated in FIG. 1, when InputSequence representing an amino acid sequence is input, the structure prediction model of AlphaFold2 outputs one 3D structure as a typical atomic structure of protein.

Incidentally, for drug discovery, prediction of an atomic structure of protein is an important elemental technique. For application to drug discovery, prediction of various atomic structures other than a typical atomic structure is needed.
Patent Literature 2: Arthur Ecoffet, et. al “MorphOT: transport-based interpolation between EM maps with UCSF ChimeraX”, (online), (Searched on Feb. 6, 2025), the Internet <academic.oup.com/bioinformatics/article/36/22-23/5528/6039110>

### SUMMARY

According to an aspect of an embodiment, a predictive control method includes acquiring first information that corresponds to a predicted structure predicted by a structure prediction model based on machine learning, and second information that is a fitting target of the first information, generating intermediate information between the first information and the second information based on the acquired first information and the acquired second information, and updating an intermediate feature amount of the structure prediction model based on the generated intermediate information.

The object and advantages of the invention will be realized and attained by means of the elements and combinations particularly pointed out in the claims.

It is to be understood that both the foregoing general description and the following detailed description are exemplary and explanatory and are not restrictive of the invention, as claimed.

### BRIEF DESCRIPTION OF DRAWINGS

FIG. 1 is a reference diagram illustrating a structure prediction model of AlphaFold2;

FIG. 2 is a schematic diagram illustrating a summary of the structure prediction model of AlphaFold2;

FIG. 3 is a diagram illustrating an image of a predictive control according to a first embodiment;

FIG. 4 is a diagram illustrating an image of an atomic structure search method according to the first embodiment;

FIG. 5A is a diagram (1) illustrating an example of an application result to which fitting by a predictive control method according to the first embodiment is applied;

FIG. 5B is a diagram (2) illustrating the example of the application result to which the fitting by the predictive control method according to the first embodiment is applied;

FIG. 6 is a diagram illustrating an image of an atomic structure search method according to a second embodiment;

FIG. 7 is a diagram illustrating an image of a difference between the atomic structure search methods according to the first embodiment and the second embodiment;

FIG. 8 is a diagram illustrating an example of an application result to which fitting by a predictive control method according to the second embodiment is applied;

FIG. 9 is a diagram illustrating an example of a functional configuration of an information processing device according to the first embodiment;

FIG. 10 is a diagram illustrating spread of electrons;

FIG. 11 is a diagram illustrating a display example of an atomic structure to be output;

FIG. 12 is a diagram illustrating a display example of coordinates of the atomic structure to be output;

FIG. 13 is a diagram illustrating an example of a flowchart of a predictive control process according to the first embodiment;

FIG. 14 is a diagram illustrating an example of a functional configuration of an information processing device according to the second embodiment;

FIG. 15 is a diagram illustrating an example of a flowchart of a predictive control process according to the second embodiment; and

FIG. 16 is a diagram illustrating a hardware configuration example.

### DESCRIPTION OF EMBODIMENTS

However, in the structure prediction model disclosed in the related art, only one typical atomic structure is output. Therefore, it is difficult to predict various atomic structures. Further, it is difficult to improve accuracy of fitting during prediction of a target structure.

Preferred embodiments will be explained with reference to accompanying drawings. Each of the embodiments is not limited to one example or aspect, and a range of numerical values or functions, a usage scene, and the like are not limited to the embodiments. In each of the embodiments, processing details can be appropriately combined within a range where the details are not contradictory to each other.

### 1. Introduction

In the related art, a technique of predicting high-dimensional structure data using a molecular structure prediction model is known.

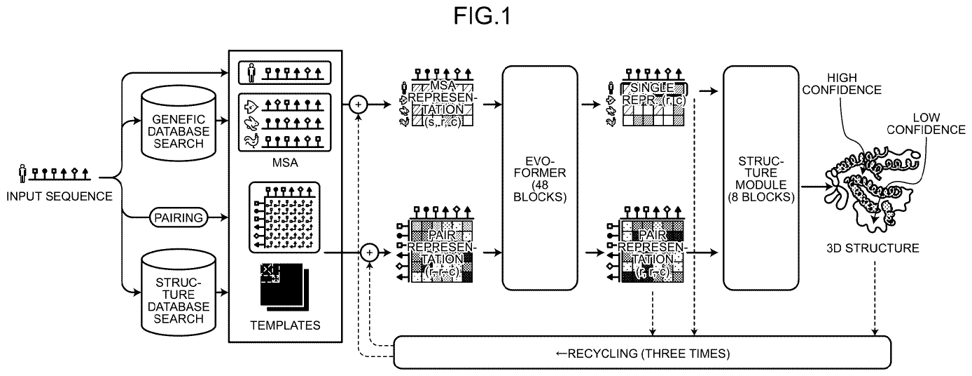

Examples of the technique in the related art of predicting high-dimensional structure data using the molecular structure prediction model include the technique disclosed in John Jumper, et. al “Highly accurate protein structure prediction with AlphaFold”, (online), (Searched on Feb. 6, 2025), the Internet <www.nature.com/articles/s41586-021-03819-2> (in particular, the schematic diagram of the structure prediction model illustrated in FIG. 1E). Although the details are not described, the structure prediction model disclosed in John Jumper, et. al “Highly accurate protein structure prediction with AlphaFold”, (online), (Searched on Feb. 6, 2025), the Internet <www.nature.com/articles/s41586-021-03819-2> is called AlphaFold2, and is a model that outputs 3D structure data when an amino acid sequence is input.

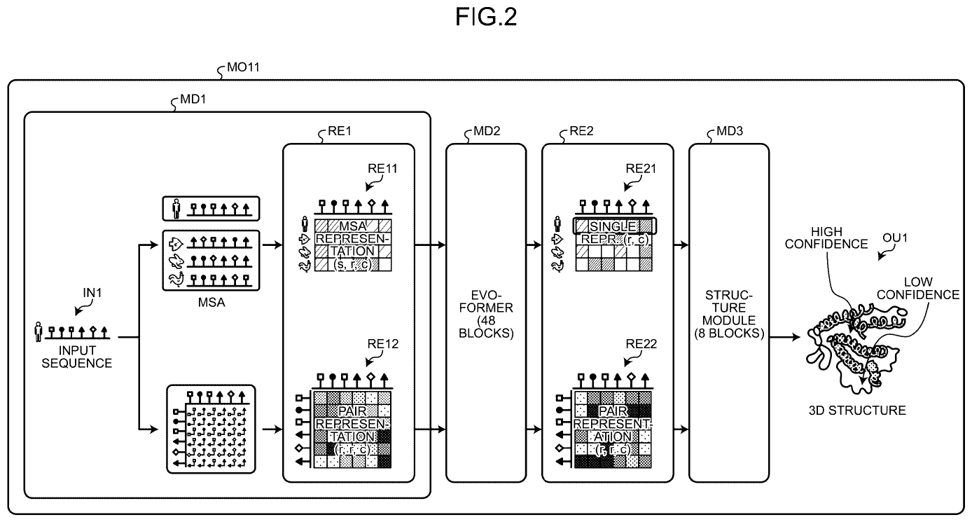

FIG. 2 is a schematic diagram illustrating a summary of the structure prediction model of AlphaFold2. A structure prediction model MO11 includes a preprocessing module MD1 that executes data shaping, a feature extraction module MD2 (corresponding to an encoder called Evoformer) that extracts a characteristic feature amount, and a structure prediction module MD3 that outputs 3D structure data.

The preprocessing module MD1 generates two types of intermediate feature amounts from an amino acid sequence IN1. One type is an array feature amount of the amino acid sequence IN1, and is a feature amount that is generated based on multiple sequence alignment (MSA). The first feature amount corresponds to MSA representation (s,r,c), and will be appropriately referred to as “first intermediate feature amount RE11”.

The other type is a spatial (structural) feature amount of the amino acid sequence IN1, and is a feature amount that is generated based on pairing of amino acid. The second feature amount corresponds to Pair representation (r,r,c), and will be appropriately referred to as “first intermediate feature amount RE12”. The two types of intermediate feature amounts generated as described above will be collectively appropriately referred to as “first intermediate feature amount RE1”. The first intermediate feature amount RE1 is an intermediate feature amount to be input to the feature extraction module MD2 (that is, an intermediate feature amount that is an input target of Evoformer).

In the feature extraction module MD2, a characteristic feature amount is extracted from each of the two types of intermediate feature amounts generated in the preprocessing module MD1. That is, two types of feature amounts are extracted. One type is a feature amount that is extracted from the first intermediate feature amount RE11. The first feature amount corresponds to Single representation (r,c), and will be appropriately referred to as “second intermediate feature amount RE21”.

The other type is a feature amount that is extracted from the first intermediate feature amount RE12. The second feature amount corresponds to Pair representation (r,r,c), and will be appropriately referred to as “second intermediate feature amount RE22”. The two types of intermediate feature amounts extracted as described above will be collectively appropriately referred to as “second intermediate feature amount RE2”. The second intermediate feature amount RE2 is an intermediate feature amount to be input to the structure prediction module MD3 (that is, an intermediate feature amount that is an input target of “Structure module”).

In the structure prediction module MD3, 3D structure data is generated from the two types of intermediate feature amounts extracted in the feature extraction module MD2.

However, in the structure prediction model disclosed in the related art, only one typical atomic structure is output. Therefore, it is difficult to predict various atomic structures.

Accordingly, in the following embodiment, the structure prediction model MO11 based on AlphaFold2 is proposed such that various atomic structures can be predicted, and a predictive control method using the structure prediction model MO11 is further proposed to improve accuracy of fitting during prediction of a target structure.

In the following embodiment, the structure prediction model MO11 is a model that outputs 3D structure data of an organic compound (for example, protein) when an amino acid sequence is input. The structure prediction model MO11 is a model that outputs, when an amino acid sequence is input, 3D structure data of an organic compound as a target structure corresponding to the input amino acid sequence. In addition, when the structure prediction model MO11 includes a plurality of layers, the structure prediction model MO11 may generate an intermediate feature amount in each of the plurality of layers. For example, the first intermediate feature amount RE1 may be generated in a first layer, and the second intermediate feature amount RE2 may be generated in a second layer.

### 2. Description of Information Processing

Next, information processing according to embodiments will be described. Description will be given using two embodiments including a first embodiment that aims to predict various atomic structures and a second embodiment that aims to improve accuracy of fitting during prediction of a target structure. The details described in the first embodiment will be appropriately omitted in the description of the second embodiment.

### (a) First Embodiment

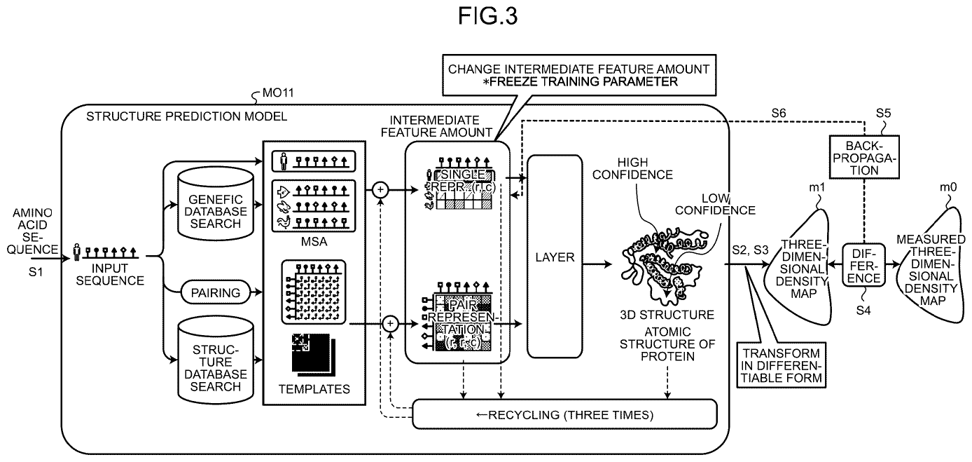

First, an image of a predictive control according to the first embodiment will be described with reference to FIG. 3. FIG. 3 is a diagram illustrating the image of the predictive control according to the first embodiment. In the predictive control process illustrated in FIG. 3, the intermediate feature amount (the first intermediate feature amount RE1 or the second intermediate feature amount RE2) of the structure prediction model is updated to be fitted to information for limiting a target atomic structure, and the atomic structure is predicted. Hereinafter, the information for limiting the atomic structure will be referred to as limiting information. In addition, in FIG. 3, the intermediate feature amounts such as the first intermediate feature amount RE1 or the second intermediate feature amount RE2 will be described without distinction.

Hereinafter, protein will be described as an example of the target, but the target is not limited to protein. For example, the target may be an organic compound other than protein. In addition, examples of the information for limiting the atomic structure of the target (limiting information) include a three-dimensional density map for limiting the atomic structure of protein. However, the limiting information is not limited to the three-dimensional density map. The three-dimensional density map may be represented in the format of, for example, three-dimensional voxel data. The three-dimensional density map is represented, for example, in a format where a three-dimensional grid having a size of nₓ×n_{y}×n_{z} includes a density value. In the following description, all of auxiliary information, intermediate auxiliary information, and target auxiliary information described below are three-dimensional density maps.

As illustrated in FIG. 3, the predictive control process includes the structure prediction model MO11. The structure prediction model MO11 described herein may be, for example, a machine learning model such as AlphaFold2 or OpenFold. In the first embodiment, the description will be given assuming that AlphaFold2 is applied to the structure prediction model MO11.

In the predictive control process, sequence information of amino acids is input to the structure prediction model MO11 (Step S1). As a result, the structure prediction model MO11 extracts the intermediate feature amount from the sequence information of amino acids, inputs the extracted intermediate feature amount to a layer, and outputs the atomic structure (three-dimensional structure) of protein as a prediction result (Step S2). The layer of the structure prediction model MO11 is a single layer for convenience of description. However, a plurality of layers may be adopted.

In the predictive control process, the atomic structure output as the prediction result is transformed into a three-dimensional density map for limiting the atomic structure in a differentiable form (Step S3).

In the predictive control process, a difference between the three-dimensional density map corresponding to the atomic structure of the prediction result and a measured three-dimensional density map is calculated (Step S4). The measured three-dimensional density map is, for example, an electron microscope (EM) image obtained by an electron microscope such as a cryo-electron microscope, or an EM image for limiting the atomic structure of protein. However, the measured three-dimensional density map is not limited thereto, and may be in the format of voxel data.

In the predictive control process, backpropagation of the difference is calculated, and an intermediate feature amount for minimizing the difference is calculated (Step S5). In the predictive control process, the intermediate feature amount of the structure prediction model MO11 is updated to the calculated intermediate feature amount (Step S6). That is, in the predictive control process, the intermediate feature amount of the structure prediction model MO11 is updated such that the difference between the three-dimensional density map corresponding to the atomic structure of the prediction result and the measured three-dimensional density map is reduced. In other words, in the predictive control process, the intermediate feature amount of the structure prediction model MO11 is updated due to a constraint from the measured three-dimensional density map.

Next, the structure prediction model MO11 predicts the atomic structure of protein using the updated intermediate feature amount. In the predictive control process, by repeating Step S2 to Step S6, various atomic structures can be predicted.

When the structure prediction model MO11 includes a plurality of layers, in the predictive control process, an intermediate feature amount to be input to any of the plurality of layers may be updated. This intermediate feature amount also corresponds to the first intermediate feature amount RE1 and the second intermediate feature amount RE2. By backpropagation, at least one of a plurality of intermediate feature amounts including the first intermediate feature amount RE1 and the second intermediate feature amount RE2 is updated. For example, among the first intermediate feature amount RE1 and the second intermediate feature amount RE2, one intermediate feature amount may be updated, or both intermediate feature amounts may be updated. Hereinafter, for convenience of description, a case where the second intermediate feature amount RE2 is updated will be described as an example.

In addition, in the predictive control process, the intermediate feature amount of the structure prediction model MO11 is updated, but a training parameter of the structure prediction model MO11 is not updated. The reason for this is that an existing training parameter is not corrupted by retraining of the structure prediction model MO11 such that loss of knowledge in the structure prediction model MO11 is avoided.

The image of the predictive control according to the first embodiment is as described above. The predictive control method according to the first embodiment is a method for searching for the atomic structure of protein using the structure prediction model MO11, in which the structure prediction model MO11 that is previously trained is used. In addition, in the predictive control method according to the first embodiment, the intermediate feature amount is updated by backpropagation based on a loss function corresponding to a desired property, and the updated intermediate feature amount is processed again in the structure prediction model MO11 on the network to generate a new structure having the desired property. The predicted structure is changed to be guided to the new structure having the target property. That is, while gradually changing the predicted structure by updating the intermediate feature amount, the structure is predicted to be finally fitted to a density map of the target structure.

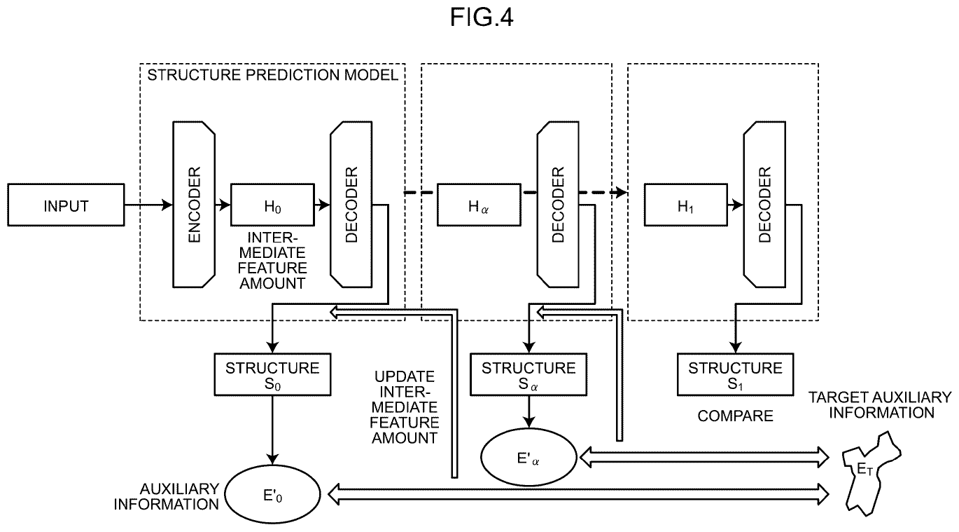

Further, an image of an atomic structure search method according to the first embodiment will be described with reference to FIG. 4. FIG. 4 is a diagram illustrating the image of the atomic structure search method according to the first embodiment. In the atomic structure search method illustrated in FIG. 4, first, an intermediate feature amount H₀ is updated based on a comparison between auxiliary information (target auxiliary information) E_{T} corresponding to the target structure and auxiliary information E′₀ corresponding to a predicted structure S₀. The updated intermediate feature amount is an intermediate feature amount H_{α}. The intermediate feature amount H_{α} is input to a decoder to generate a predicted structure Sa. The predicted structure S_{α} is transformed to obtain auxiliary information E′_{α}. Next, the intermediate feature amount Ha is updated based on a comparison between the target auxiliary information E_{T} and the auxiliary information E′_{α} corresponding to the predicted structure Sa. The updated intermediate feature amount is an intermediate feature amount H₁. The intermediate feature amount H₁ is input to a decoder to generate a predicted structure S₁. The predicted structure S₁ is transformed to obtain auxiliary information E′₁. The intermediate feature amount H₁ is updated based on a comparison between the target auxiliary information E_{T} and the auxiliary information E′₁ corresponding to the predicted structure S₁.

In the first embodiment, the predictive control method of predicting the atomic structure (the target structure) of the target based on the indirect auxiliary information (corresponding to the three-dimensional density map) using the structure prediction model MO11 is proposed. Proposed is the predictive control method of updating the intermediate feature amount (for example, the first intermediate feature amount RE1 or the second intermediate feature amount RE2) of the structure prediction model MO11 to be fitted to the three-dimensional density map of the target structure.

In the predictive control method according to the first embodiment, from the predicted structure of the structure prediction model MO11 as a starting point, error between the predicted structure and the target structure is calculated using the target auxiliary information as a hint, and the intermediate feature amount of the structure prediction model MO11 is updated for fitting by backpropagation.

However, in the predictive control method according to the first embodiment, it may be difficult to improve the accuracy of fitting during the prediction of the target structure. For example, the fitting process follows an unnatural deformation process that passes through an intermediate feature amount deviating from a training range of the structure prediction model MO11, and it may be difficult to complete the fitting. For example, the intermediate state of the fitting may deviate from the training range, or the starting point structure may largely deviate from the target structure. In this case, it may be difficult to complete the fitting.

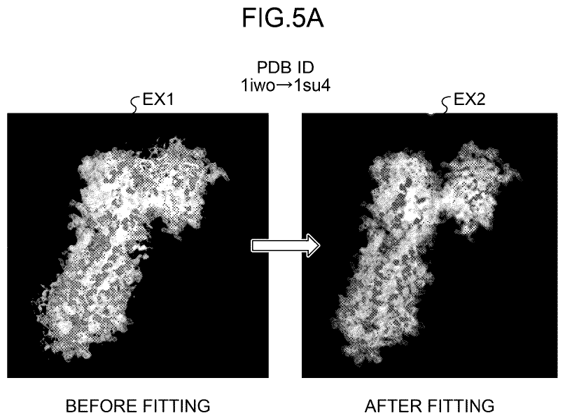

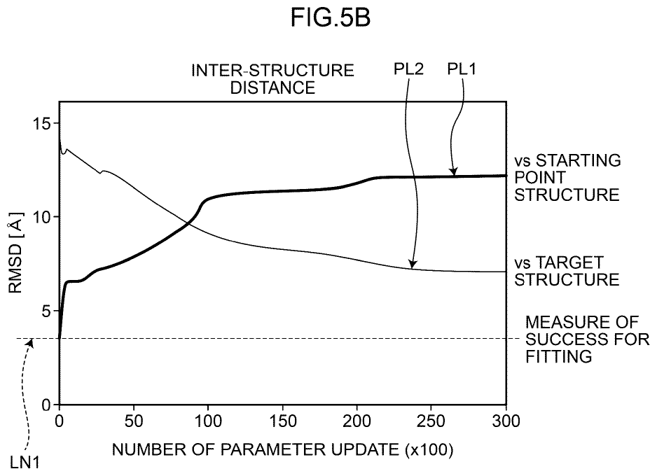

FIGS. 5A and 5B are diagrams illustrating an example of an application result to which the fitting by the predictive control method according to the first embodiment is applied. The results illustrated in FIGS. 5A and 5B are results of referring to a known protein structure registered in a database called a protein data bank (PDB), and are used for referring to a protein structure similar to the starting point structure (liwo) and a protein structure similar to the target structure (lsu4). liwo and lsu4 are proteins having structures that have the same amino acid sequence and different shapes.

In a result EX1 and a result EX2 illustrated in FIG. 5A, a white portion is a portion where electron density is visualized, and a gray portion is a portion where the predicted structure is visualized. The result EX1 is data before fitting, and the result EX2 is data after fitting. It can be seen from the result EX1 and the result EX2 that, in the data before fitting, there is a large deviation from the white portion, and fitting was not sufficiently executed.

FIG. 5B illustrates data where an inter-structure distance is plotted. In FIG. 5B, an index of the vertical axis is RMSD, and an index of the horizontal axis is the number of updates. A plot PL1 is a difference from the starting point structure, and a plot PL2 is a difference from the target structure. A dotted line LN1 is a threshold representing a measure of success for fitting. When a plot PL2 reaches the dotted line LN1, it represents that fitting was able to be sufficiently executed. In FIG. 5B, the plot PL2 tends to decrease, but does not reach the dotted line LN1. Therefore, it can be seen that fitting was not sufficiently executed.

It can be seen from the plot PL1 that, in the state before fitting, the predicted structure does not completely match with the starting point structure but substantially matches with the starting point structure. In addition, as the fitting progresses such that the predicted structure approaches the target structure, the predicted structure deviates from the starting point structure. Therefore, it can be seen that the plot PL1 tends to increase in contrast to the plot PL2.

When a difference between the starting point structure and the target structure is large, a deviation of the inter-structure distance increases, and thus the fitting is not likely to be sufficiently executed.

Accordingly, a predictive control method using the structure prediction model MO11 is further proposed to improve accuracy of fitting during prediction of a target structure.

### (b) Second Embodiment

In the second embodiment, proposed is a predictive control method of generating an intermediate structure between the starting point structure and the target structure in an auxiliary information space in a pseudo manner and guiding the predicted structure stepwise to the intermediate structure for improving the accuracy of fitting by a natural deformation process.

In the predictive control method according to the first embodiment, the target auxiliary information is directly referred to. Therefore, in the comparison to the starting point structure, as the deviation increases, prediction accuracy decreases. On the other hand, in the second embodiment, an intermediate structure more similar to the starting point structure than the target structure is provided, and the predicted structure is gradually updated as compared to the intermediate structure and is guided to the intermediate structure. That is, a density map of the intermediate structure (hereinafter, appropriately referred to as “intermediate auxiliary information”) is generated. The number of pieces of the intermediate auxiliary information is not limited, and is at least one.

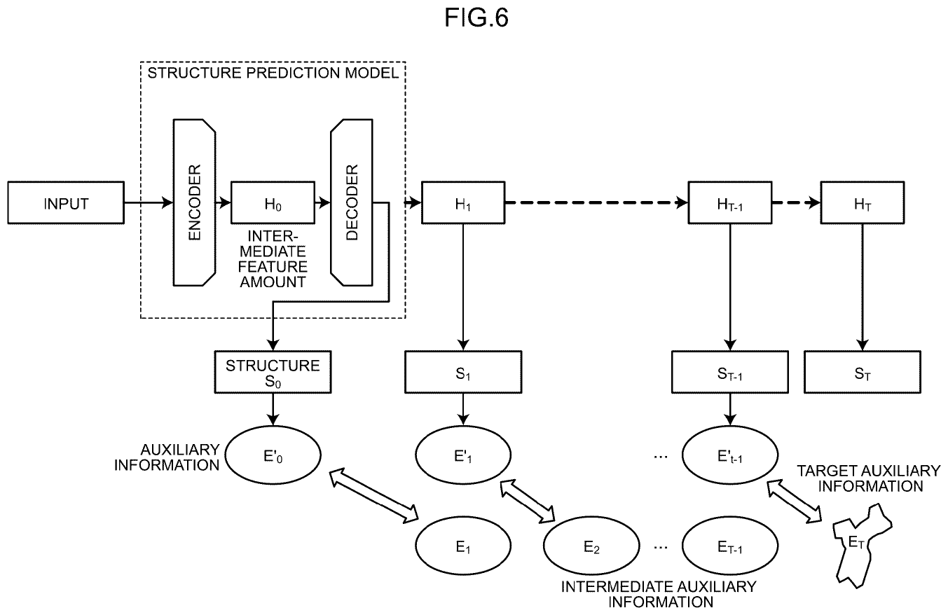

Further, an image of an atomic structure search method according to the second embodiment will be described with reference to FIG. 6. FIG. 6 is a diagram illustrating the image of the atomic structure search method according to the second embodiment. As an input, the amino acid sequence IN1 that is a prediction target and the target auxiliary information E_{T} are used. The predicted structure S₀ is a predicted structure of initial prediction, and the auxiliary information E′₀ is auxiliary information corresponding to the predicted structure of the initial prediction.

In the atomic structure search method illustrated in FIG. 6, first, intermediate auxiliary information E₁ to intermediate auxiliary information E_{T-1} are generated between the target auxiliary information E_{T} and the auxiliary information E′₀. The intermediate feature amount H₀ is updated based on a comparison between the intermediate auxiliary information E₁ and the auxiliary information E′₀. The updated intermediate feature amount is an intermediate feature amount H₁. The intermediate feature amount H₁ is updated based on a comparison between the intermediate auxiliary information E₂ and the auxiliary information E′₁.

This way, in FIG. 6, from a predicted structure Sₜ₋₁ as the starting point structure, the auxiliary information E′₀ corresponding to the predicted structure Sₜ₋₁ is fitted to the intermediate auxiliary information Eₜ to generate a predicted structure Sₜ. By repeating this process T times, a predicted structure S_{T} is generated. The predicted structure is fitted to the intermediate auxiliary information similar to the auxiliary information corresponding to the starting point structure, and once the fitting is completed, the predicted structure is fitted to the next intermediate auxiliary information. By repeating this process in order, the predicted structure is finally fitted to the target auxiliary information E_{T}.

This way, by providing the intermediate auxiliary information E₁ to the intermediate auxiliary information E_{T-1} to repeat the fitting, the predicted structure S_{T} that is finally fitted to the target auxiliary information E_{T} is generated.

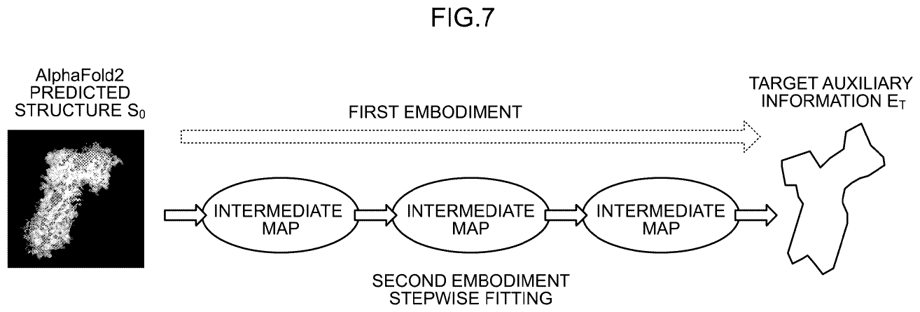

FIG. 7 is a diagram illustrating an image of a difference between the atomic structure search methods according to the first embodiment and the second embodiment. In the atomic structure search method according to the first embodiment, the target auxiliary information E_{T} is directly predicted from the predicted structure S₀. On the other hand, in the atomic structure search method according to the second embodiment, the density map of the intermediate structure between the predicted structure S₀ and the target structure is generated in a pseudo manner, and the predicted structure is finally guided to the target structure while being fitted to the intermediate structure in order from the intermediate structure similar to the predicted structure S₀.

Hereinafter, details of a generation condition of the intermediate auxiliary information will be described. The intermediate auxiliary information is generated based on a predetermined condition. In the auxiliary information space, an index representing ease of reaching the target auxiliary information E_{T} from the intermediate auxiliary information Eₜ is an index d(Eₜ, E_{T}). As the value of the index d(Eₜ, E_{T}) decreases, the predicted structure approaches and is more likely to reach the target auxiliary information E_{T}. For example, when the predicted structure that is predicted in the structure prediction model MO11 at the present time is the predicted structure Sₜ, the index d(Eₜ, E_{T}) represents the ease of reaching the final target structure from the predicted structure Sₜ at the present time. The index d(Eₜ, E_{T}) merely defines the generation condition of the intermediate auxiliary information, and does not need to be directly calculated for the target.

An example of the index d(Eₜ, E_{T}) will be described. The index d(Eₜ, E_{T}) is represented by, for example, Expression (1). ρ^{tag}_{Ng} represents a density map of the target structure, and ρ^{tag}_{Ng} represents a density map of the predicted structure. A cross-correlation between the density maps (hereinafter, appropriately referred to as “cross-correlation C1”) represents the degree of similarity between the density maps. As the degree of similarity between the density map of the target structure and the density map of the predicted structure increases, the predicted structure is more likely to reach the target structure.

$$Expression ( 1 )$$

$${L}_{N} = 1 - Cross - correlation between Density Maps = 1 - \frac{{\sum }_{g}^{{N}^{3}} {ρ}_{Ng}^{pred} {ρ}_{Ng}^{targ}}{\sqrt{{\sum }_{g}^{{N}^{3}} {( {ρ}_{Ng}^{pred} )}^{2}} \sqrt{{\sum }_{g}^{{N}^{3}} {( {ρ}_{Ng}^{targ} )}^{2}}}$$

In addition, the index d(Eₜ, E_{T}) is represented by, for example, a Wasserstein distance. For example, the index d(Eₜ, E_{T}) is represented by a Wasserstein distance W²₂(Eₜ, Eₜ) between the density map Eₜ and the density map Eₜ represented by density values on three-dimensional voxels.

Further, the index d(Eₜ, E_{T}) is represented by inter-structure distances that are corrected by a structure probability estimated by a structure probability distribution estimation method (for example, a method disclosed in Kimihiro Yamazaki, et. al “An Auto-Encoder to Reconstruct Structure with Cryo-EM Images via Theoretically Guaranteed Isometric Latent Space, and its Application for Automatically Computing the Conformational Pathway”, (online), (Searched on Feb. 6, 2025), the Internet, <conferences.miccai.org/2023/papers/055-Paper2173.html>) in the auxiliary information space. In this method, when a path that reaches the target structure is estimated, whether a path having a high probability is present is calculated.

The generation condition is that, even when the index d(Eₜ, E_{T}) is any value, the index d(Eₜ, E_{T}) satisfies Expression (2). That is, the generation condition is that the next intermediate auxiliary information to be used for fitting is in a state of being likely to reach the final target auxiliary information E_{T}.

$$d ( {E}_{i} , {E}_{T} ) > d ( {E}_{j} , {E}_{T} ) , i < j < T \qquad Expression ( 2 )$$

“The state of being likely to reach” in the present embodiment refers to a state of being more likely to reach the target than the state at the present time. Therefore, the ease of reaching in the present embodiment is not limited to closeness in terms of a distance or structural closeness. The ease of reaching in the present embodiment includes, for example, a path where the density maps become more distant from each other as a cross-correlation therebetween (a path where the density maps become temporarily more distant from each other although they finally approach each other).

Hereinafter, details of a method of generating the intermediate auxiliary information will be described. Based on the starting point structure (including the predicted structure of the initial prediction), all pieces of the intermediate auxiliary information may be collectively generated, or the intermediate auxiliary information may be dynamically generated in the process of stepwise fitting. For example, in a case where the intermediate auxiliary information is dynamically generated, when the example of FIG. 6 is used, after generating the auxiliary information E′₁, the intermediate auxiliary information E₂ to the intermediate auxiliary information E_{T-1} may be generated between the target auxiliary information E_{T} and the auxiliary information E′₁, or only the next intermediate auxiliary information E₂ to be used for fitting may be generated.

When all pieces of the intermediate auxiliary information are collectively generated, for example, a method of generating the intermediate auxiliary information based on linear interpolation between the density maps (for example, a method represented by Expression (3)) or a method of generating the intermediate auxiliary information based on density interpolation that is based on optimal transport (for example, a method disclosed in Kimihiro Yamazaki, et. al “An Auto-Encoder to Reconstruct Structure with Cryo-EM Images via Theoretically Guaranteed Isometric Latent Space, and its Application for Automatically Computing the Conformational Pathway”, (online), (Searched on Feb. 6, 2025), the Internet, <conferences.miccai.org/2023/papers/055-Paper2173.html>) may be used. The latter method is based on the path that is present when approaching the target auxiliary information, and can execute more natural interpolation (more natural approach method to the target auxiliary information) than the former method.

$${E}_{t} = \frac{t}{T} {E}_{T} + \frac{T - t}{T} {E}_{0}^{′} \qquad Expression ( 3 )$$

When the intermediate auxiliary information is dynamically generated, for example, a method of generating the intermediate auxiliary information based on linear interpolation between the auxiliary information E′ₜ₋₁ and the target auxiliary information E_{T} (for example, a method represented by Expression (4)) or a method of generating a plurality of intermediate auxiliary information candidates as the intermediate auxiliary information around the auxiliary information E′ₜ₋₁ and setting, as the intermediate auxiliary information Eₜ, the intermediate auxiliary information candidate for minimizing the index d(Eₜ, E_{T}) may be used.

$${E}_{t} = \frac{1}{T - t + 1} {E}_{T} + \frac{T - t}{T - t + 1} {E}_{t - 1}^{′} \qquad Expression ( 4 )$$

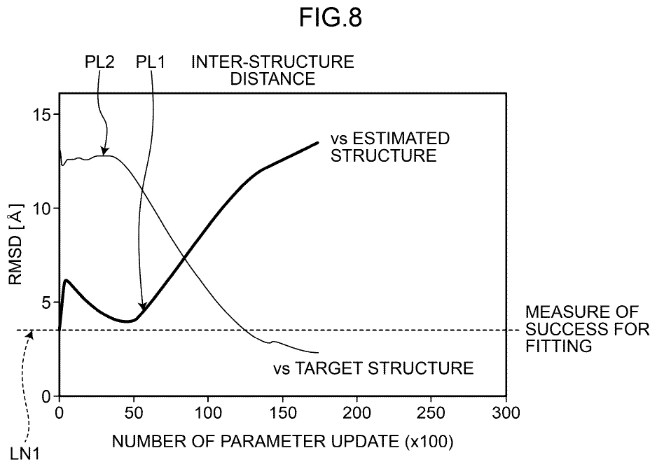

FIG. 8 is a diagram illustrating an example of an application result to which fitting by a predictive control method according to the second embodiment is applied. FIG. 8 illustrates a comparison to FIG. 5B. Unlike FIG. 5B, the plot PL2 reaches the dotted line LN1. Therefore, it can be seen that fitting was able to be sufficiently executed. In addition, it can also be seen that fitting is fast. By providing appropriate intermediate auxiliary information, improvement of final fitting can be expected.

As described above, in the predictive control method according to the second embodiment, the intermediate feature amount (in particular, the second intermediate feature amount RE2) of the structure prediction model MO11 based on machine learning is updated, and the target structure is predicted based on the target auxiliary information E_{T}. In addition, in the predictive control method according to the second embodiment, interpolation between the auxiliary information E′ₜ corresponding to the predicted structure Sₜ predicted from the structure prediction model MO11 at a certain time and the target auxiliary information E_{T} is executed by the pseudo intermediate auxiliary information. In addition, in the predictive control method according to the second embodiment, the quality of fitting is improved by sequentially executing fitting with the interpolated intermediate auxiliary information as a target. In addition, in the predictive control method according to the second embodiment, the prediction accuracy of the final target structure is improved by executing fitting stepwise to the density map of the intermediate structure (intermediate auxiliary information) generated based on the density map of the predicted structure Sₜ (auxiliary information E′ₜ) predicted by the structure prediction model MO11 and the density map of the target structure (target auxiliary information E_{T}).

### 3. Configuration of Information Processing Device

### First Embodiment

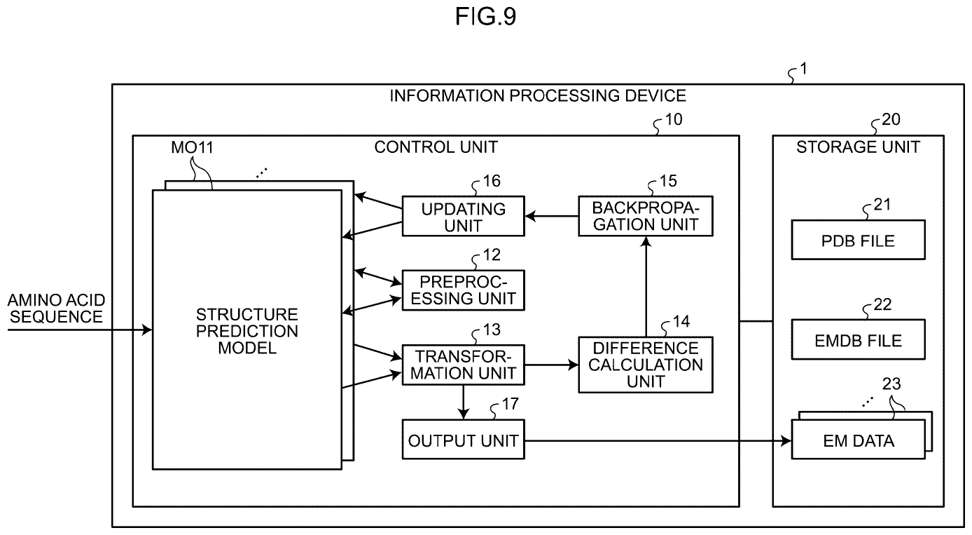

FIG. 9 is a diagram illustrating an example of a functional configuration of an information processing device according to the first embodiment. The information processing device 1 illustrated in FIG. 9 is an example of a computer that executes the predictive control process. As illustrated in FIG. 9, the information processing device 1 includes a control unit 10 and a storage unit 20.

The storage unit 20 includes a protein data bank (PDB) file 21, an electron microscope data bank (EMDB) file 22, and EM data 23.

The PDB file 21 is a file where information regarding a three-dimensional atomic structure of protein is accumulated. The PDB file 21 includes, for example, information regarding atomic structures of three monomers forming protein, and also includes information regarding a chain to which each atom belongs. The PDB file 21 may be acquired from PDB on the WEB.

The EMDB file 22 is a file where a group of EM images that are obtained by an electron microscope such as a cryo-electron microscope and limit the three-dimensional atomic structure of protein is accumulated. The EM image has the same definition as, for example, the three-dimensional density map. The EMDB file 22 may be acquired from EMDB on the WEB.

The EM data 23 is the three-dimensional density map of the atomic structure predicted by the predictive control process. The EM data 23 is stored in the storage unit 20 by an output unit 17 described below.

The control unit 10 includes a plurality of structure prediction models MO11, a preprocessing unit 12, a transformation unit 13, a difference calculation unit 14, a backpropagation unit 15, an updating unit 16, and the output unit 17.

The structure prediction model MO11 predicts the atomic structure forming protein from sequence information of amino acids. Each of the structure prediction models MO11 corresponds to one monomer. When the sequence information of amino acids is a multimer, each of the structure prediction models MO11 predicts an atomic structure of a monomer forming protein for each chain. In the first embodiment, a case where the protein of the target is a trimer will be described.

The preprocessing unit 12 executes preprocessing of the predictive control.

In an example of first preprocessing, the preprocessing unit 12 acquires, by point cloud registration, three rigid transformations that match with each chain of atomic structures of predicted three monomers (trimer) using the PDB file 21. The reason for this is to acquire rigid transformations corresponding to scattered chains. The PDB file 21 includes information regarding atomic structures of three monomers forming protein, and also includes information regarding a chain to which each atom belongs. Accordingly, by using the PDB file 21, the preprocessing unit 12 can easily execute the point cloud registration for each chain of the atomic structures of each of the predicted trimer, and three rigid transformations suitable for each chain can be acquired. The preprocessing unit 12 applies the acquired rigid transformation to each chain of the atomic structure of each of the predicted three monomers, and combines the three monomers into one multimer.

In an example of second preprocessing, the preprocessing unit 12 determines the rigid transformations as the best match between the combined multimer and the three-dimensional density map of the target in the EMDB file 22. That is, the preprocessing unit 12 determines the rigid transformations used for executing registration between the atomic structure and the three-dimensional density map of the target. An expression for the registration is represented by, for example, Expression (5). Rc and tc are the rigid transformations representing rotation and centroid (translation). xca and x′ca represent original atomic coordinates of an atom a and atomic coordinates of the atom a after the rigid transformation.

$${x}_{ca}^{′} = {R}_{c} {x}_{c a} + {t}_{c} \qquad Expression ( 5 )$$

For example, in the second preprocessing, the preprocessing unit 12 executes a first step and a second step. In the first step, using the three-dimensional density map of the target, the preprocessing unit 12 executes centroid alignment (translation) and principal component axis alignment (rotation) for the combined multimer to determine tc and Rc. In the second step, the preprocessing unit 12 finely adjusts the registration based on Expression (5) using Rc and tc as variables and using xca as a constant (fixed) to determine tc and Rc. That is, in the second preprocessing, without using the information regarding a chain to which an atom of each monomer originally belongs, a method of translating and rotating a rigid body that is one multimer where the monomers are combined to acquire the best match with the three-dimensional density map of the target is acquired. As a result, the preprocessing unit 12 can execute the registration between the atomic structure and the three-dimensional density map of the target.

When the preprocessing ends, the preprocessing unit 12 executes forward propagation of the layer of the structure prediction model MO11 for each chain. An expression for the forward propagation of the layer is represented by, for example, Expression (6). rci represents the intermediate feature amount of each chain. i represents an index representing each chain. xca represents atomic coordinates of the predicted atomic structure. When the preprocessing unit 12 executes the forward propagation of the layer, the intermediate feature amount of the structure prediction model MO11 is not updated.

$${x}_{c a} = StructureModule ( {r}_{c i} ) \qquad Expression ( 6 )$$

In the second preprocessing, the rigid transformations for obtaining the best match between the combined multimer and the three-dimensional density map of the target are determined in the two steps including the first step and the second step. However, the second preprocessing is not limited thereto, and the rigid transformations for obtaining the best match between the combined multimer and the three-dimensional density map of the target may be determined using a genetic algorithm.

The transformation unit 13 transforms the atomic structure to be output as the prediction result from the structure prediction model MO11 into a three-dimensional density map (EM) for limiting the atomic structure in a differentiable form.

For example, the transformation unit 13 applies the rigid transformations acquired in the preprocessing unit 12 to the atomic structure of the prediction result to acquire an atomic structure of one multimer (trimer). That is, the transformation unit 13 acquires the atomic structure of one multimer (trimer) from the atomic structure of the prediction result using Expression (5).

Using an interpolation expression satisfying the density conservation law, the transformation unit 13 transforms the atomic structure of the trimer of the prediction result into the three-dimensional density map for limiting the atomic structure. The interpolation expression satisfying the density conservation law is represented, for example, by Expression (7), Expression (8), and Expression (9).

dN used in Expression (7) represents the length of one piece of voxel. x′Ngk and x′cak represent a k component of coordinates of a vertex of the voxel and a k component of the atomic coordinates, respectively. uNgcak represents a normalized distance between the atom a and a vertex g of the voxel.

$${u}_{Ngcak} = \frac{\vert {x}_{cak}^{′} - {x}_{Ngk}^{′} \vert}{{d}_{N}} \qquad Expression ( 7 )$$

uNgcak used in Expression (8) represents the normalized distance between the atom a and the vertex g of the voxel that is the calculation result of Expression (7). pNgcak represents an existence probability of the atom a in the vertex g of the voxel.

$${ρ}_{Ngcak} = \{ \begin{matrix}\frac{1}{8} {u}_{Ngcak}^{3} - \frac{3}{8} {u}_{Ngcak}^{2} + \frac{1}{2} & if {u}_{Ngcak} < 2 \\ 0 & otherwise\end{matrix} \qquad Expression ( 8 )$$

na used in Expression (9) represents an atomic number of the atom a. pNgcak represents an existence probability of the atom a in the vertex g of the voxel that is the calculation result of Expression (8). pNgpred represents the calculated three-dimensional density map.

$${p}_{Ng}^{pred} = {\sum }_{c} {\sum }_{a} {n}_{a} {\prod }_{k} {p}_{Ngcak} \qquad Expression ( 9 )$$

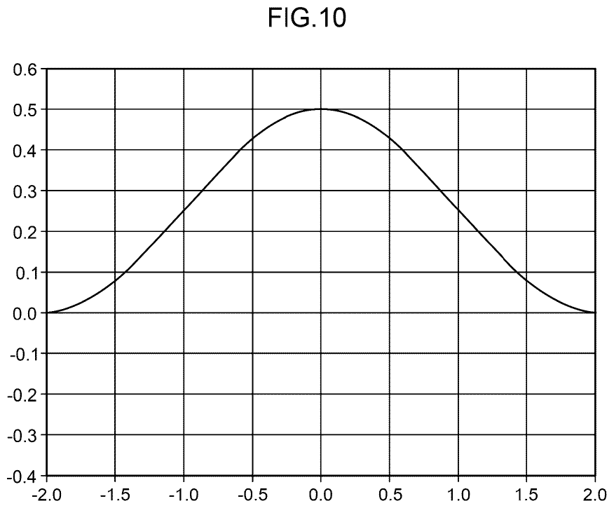

Here, FIG. 10 illustrates a relationship between the distance uNgcak between the atom a and the vertex g of the voxel represented by Expression (7) and the existence probability PNgcak of the atom a in the vertex g of the voxel represented by Expression (8). FIG. 10 is a diagram illustrating spread of electrons. The x-axis of a graph illustrated in FIG. 10 represents a value group obtained from an expression from which an absolute value of a numerator illustrated on the right side of Expression (7) is removed. That is, the x-axis represents the value group obtained by normalizing a degree to which the atom is close to the vertex of the voxel. The y-axis represents the existence probability PNgcak of the atom a in the vertex g of the voxel represented by Expression (8). It can be seen from this graph that, as the atom approaches the vertex of the voxel (as the value of the x-axis approaches 0), the existence probability of electrons increases, and as the atom becomes distant from the vertex of the voxel (as the value of the x-axis becomes distant from 0), the existence probability of electrons decreases. When the value of the x-axis is more than “2” or is less than “−2”, the existence probability of the electrons of the y-axis is “0”. Accordingly, using the existence probability PNgcak of the atom a in the vertex g of the voxel acquired in Expression (7) and Expression (8), the transformation unit 13 can transform the atomic structure into a smooth image (three-dimensional density map) where the atoms look hazy (concept of a filter). That is, the transformation unit 13 implements the transformation processing where the spread of electrons and the concept of the filter are integrated into one.

This way, using Expression (7), Expression (8), and Expression (9), the transformation unit 13 transforms the atom a of the trimer of the prediction result into the three-dimensional density map pNgpred for limiting the atom a. Expression (7), Expression (8), and Expression (9) are transformation functions from the atomic structure into the three-dimensional density map for limiting the atomic structure, and are differentiable transformation functions.

The difference calculation unit 14 calculates a difference between the three-dimensional density map corresponding to the atomic structure of the trimer of the prediction result and the three-dimensional density map of the target in the EMDB file 22. For example, by acquiring the cross-correlation, the difference calculation unit 14 calculates a difference between the three-dimensional density map for limiting the atomic structure of the prediction result and the three-dimensional density map of the target. An expression for acquiring the cross-correlation is represented, for example, by Expression (10) below.

pNgpred used in Expression (10) represents the three-dimensional density map calculated from Expression (7), Expression (8), and Expression (9). pNgtarg represents the three-dimensional density map of the target. LN represents the value of the cross-correlation between the three-dimensional density maps. g and N represent the vertex of the voxel and the number of divisions of the voxel, respectively.

$${L}_{N} = 1 - \frac{{\sum }_{g}^{{N}^{3}} {ρ}_{Ng}^{pred} {ρ}_{Ng}^{targ}}{\sqrt{{\sum }_{g}^{{N}^{3}} {( {ρ}_{Ng}^{pred} )}^{2}} \sqrt{{\sum }_{g}^{{N}^{3}} {( {ρ}_{Ng}^{targ} )}^{2}}} \qquad Expression ( 10 )$$

This way, using Expression (10), the difference calculation unit 14 calculates a difference between the three-dimensional density map corresponding to the atomic structure of the atom a of the trimer of the prediction result and the three-dimensional density map of the target. Expression (10) is a differentiable expression. The expression for calculating the difference is described as the expression for acquiring the cross-correlation, but is not limited thereto. As the expression for calculating the difference, an L2 norm or an L1 norm may also be used.

In addition, using the calculated difference, the difference calculation unit 14 calculates an objective function representing a constraint for the atomic structure of the trimer of the prediction result. Further, the difference calculation unit 14 adds a desired constraint of protein to the calculated objective function to calculate a final objective function. For example, the difference calculation unit 14 calculates the final objective function using Expression (11) below.

LN used in Expression (11) is a value calculated in Expression (10). Lboundlength and Lboundangle are examples of objective functions for maintaining a distance and an angle of a peptide bond, respectively. N represents the number of divisions of the voxel. Ltotal represents the final objective function.

$${L}_{total} = \frac{{\sum }_{N = 8}^{128} {N}^{- 1} {L}_{N}}{{\sum }_{N = 8}^{128} {N}^{- 1}} + {β}_{1} {L}_{b ondlength} + {β}_{2} {L}_{b ondangle} \qquad Expression ( 11 )$$

A first expression of the right side represented by Expression (11) is a main objective function. In the first expression, the difference is smoothly reduced using a density mixing technique of area divisions.

In addition, using Expression (11), the difference calculation unit 14 calculates an objective function representing a constraint for the atom a of the trimer of the prediction result. Expression (11) is a differentiable expression. The constraints to be added are described as, for example, the distance and the angle of the peptide bond, but are not limited thereto. For example, an excluded volume effect, a disulfide bond length, or a hydrogen bond length may be used. In short, the difference calculation unit 14 may add, as options, constraints that protein is supposed to have to the main objective function.

The backpropagation unit 15 calculates backpropagation of the difference. For example, the backpropagation unit 15 calculates backpropagation of the difference using Expression (12) to Expression (17) below. In Expression (12), Ltotal in Expression (11) is differentiated with respect to LN. In Expression (13), LN in Expression (10) is differentiated with respect to PNgpred. In Expression (14), PNgpred in Expression (9) is differentiated with respect to PNgcak. In Expression (15), PNgcak in Expression (8) is differentiated with respect to uNgcak. In Expression (16), uNgcak in Expression (7) is differentiated with respect to x′cak. In Expression (17), LN in Expression (10) is differentiated with respect to x′cak.

$$\frac{\partial {L}_{t otal}}{\partial {L}_{N}} = \frac{{N}^{- 1}}{{\sum }_{N = 8}^{128} {N}^{- 1}} \qquad Expression ( 12 )$$

$$\frac{\partial {L}_{N}}{\partial {ρ}_{Ng}^{pred}} = \frac{( {ρ}_{Ng}^{pred} / {\sum }_{g}^{{N}^{3}} {( {ρ}_{Ng}^{pred} )}^{2} ) {\sum }_{g}^{{N}^{3}} {ρ}_{Ng}^{pred} {ρ}_{Ng}^{targ} - {ρ}_{Ng}^{targ}}{\sqrt{{\sum }_{g}^{{N}^{3}} {( {ρ}_{Ng}^{pred} )}^{2}} \sqrt{{\sum }_{g}^{{N}^{3}} {( {ρ}_{Ng}^{targ} )}^{2}}} \qquad Expression ( 13 )$$

$$\frac{\partial {ρ}_{g}^{pred}}{\partial {p}_{Ngcak}} = {n}_{a} {\prod }_{{k}^{′} \neq k} {p}_{{Ngcak}^{′}} \qquad Expression ( 14 )$$

$$\frac{\partial {p}_{Ngcak}}{\partial {u}_{Ngcak}} = \{ \begin{matrix}\frac{3}{8} {u}_{Ngcak}^{2} - \frac{3}{4} {u}_{Ngcak} & if {u}_{Ngcak} < 2 \\ 0 & otherwise\end{matrix} \qquad Expression ( 15 )$$

$$\frac{\partial {u}_{Ngcak}}{\partial {x}_{cak}^{′}} = \frac{sgn ( {x}_{cak}^{′} - {x}_{Ngk}^{′} )}{{d}_{N}} \qquad Expression ( 16 )$$

$$\frac{\partial {L}_{N}}{\partial {x}_{cak}^{′}} = {\sum }_{g} \frac{\partial {L}_{N}}{\partial {ρ}_{Ng}^{p r e d}} \frac{\partial {ρ}_{N g}^{p r e d}}{\partial {p}_{Ngcak}} \frac{\partial {p}_{Nqcak}}{\partial {u}_{Ngcak}} \frac{\partial {u}_{Ngcak}}{\partial {x}_{cak}^{′}} \qquad Expression ( 17 )$$

In addition, the backpropagation unit 15 calculates the intermediate feature amount for each chain using a gradient acquired by the backpropagation of the difference. For example, the backpropagation unit 15 calculates the intermediate feature amount for each chain from Expression (18) below using the gradient acquired by the backpropagation of the difference (Expression (17)). rci in Expression (18) represents the intermediate feature amount of each chain. i represents an index representing each chain. In addition, δxca/δrci is obtained by differentiating the atomic coordinates xca in Expression (6) with respect to the intermediate feature amount rci of each chain.

$${γ}_{c i} ← {r}_{c i} - η {\sum }_{a} \frac{\partial L}{\partial {x}_{c a}} \frac{\partial {x}_{c a}}{\partial {r}_{c i}} \qquad Expression ( 18 )$$

The updating unit 16 updates the intermediate feature amount for each chain to the structure prediction model MO11 corresponding to each chain. That is, the updating unit 16 updates the intermediate feature amount of the structure prediction model MO11 to reduce a difference between the atomic structure of the prediction result and the atomic structure of the target. In other words, the updating unit 16 updates the intermediate feature amount of the structure prediction model MO11 due to a constraint from the three-dimensional density map of the target.

The output unit 17 stores the three-dimensional density map (EM) transformed by the transformation unit 13 in the storage unit 20 as the EM data 23.

### Display Example of Atomic Structure to be Output

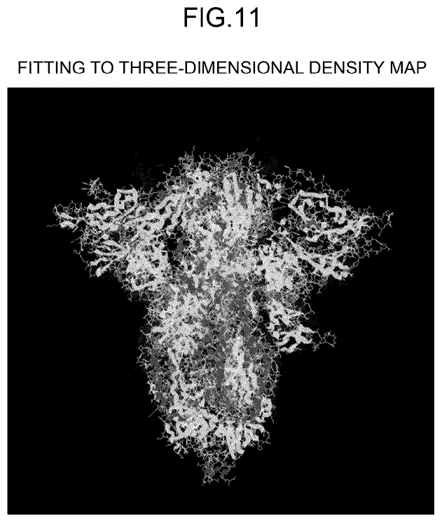

FIG. 11 is a diagram illustrating a display example of the atomic structure to be output. In the image illustrated in FIG. 11, the atomic coordinates x′ca (refer to Expression (9)) of the atomic structure output from the structure prediction model MO11 are displayed on a screen. The atomic coordinates x′ca of the atomic structure are the atomic coordinates after the rigid transformation of each chain. By operating the atomic coordinates of the atomic structure displayed on the screen, a user can use fitting (adjustment) of the three-dimensional density map.

In the first embodiment, the limiting information for limiting the atomic structure is described as the three-dimensional density map (EM image). However, the limiting information for limiting the atomic structure is not limited to the three-dimensional density map (EM image) obtained by an electron microscope, and may be a three-dimensional density map analyzed by an X-ray. In addition, the limiting information for limiting the atomic structure may be coordinates of an electronic structure.

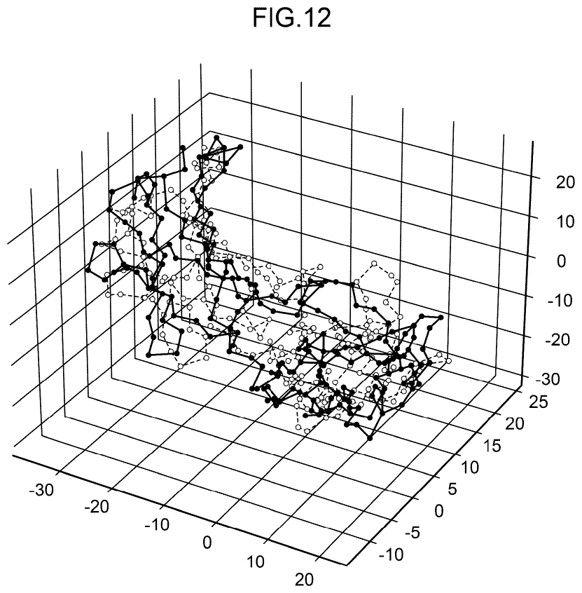

Here, when the limiting information for limiting the atomic structure is the coordinates of the electronic structure, a display example of the atomic structure to be output will be described with reference to FIG. 12. FIG. 12 is a diagram illustrating a display example of the coordinates of the atomic structure to be output. In the image illustrated in FIG. 12, the coordinates of the atomic structure output from the structure prediction model MO11 are displayed on a screen (solid line). The coordinates of the atomic structure are the atomic coordinates after the rigid transformation of each chain. Further, in the image illustrated in FIG. 12, the coordinates of the limiting information of the target are displayed on the screen (broken line). When the limiting information for limiting the atomic structure is coordinates of an electronic structure, the limiting information of the target may be obtained from the PDB file 21. A user can use the limiting information of the target such that the coordinates (solid line) of the atomic structure that are output from the structure prediction model MO11 and displayed on the screen are aligned with the coordinates (broken line) of the limiting information of the target.

In addition, in FIG. 12, only carbon atoms (Ca) present at the center of each amino acid are displayed. The number of Cα is about 1/10 of the number of all the atoms. The coordinates of the atomic structure and the structure of the line output from the structure prediction model MO11 include information regarding not only the displayed Ca but also all the atoms. On the other hand, the coordinates of the limiting information of the target and the structure of the line are obtained from the PDB file 21, and also include the information regarding the atoms other than Ca. However, only Ca is used as the limiting information. That is, even when the atomic structure that can be used as the limiting information is only 1/10 of all the atoms, a user can restore the information regarding all the atoms by using the atomic structure output from the structure prediction model MO11 as a hint.

### Flowchart of Predictive Control Process

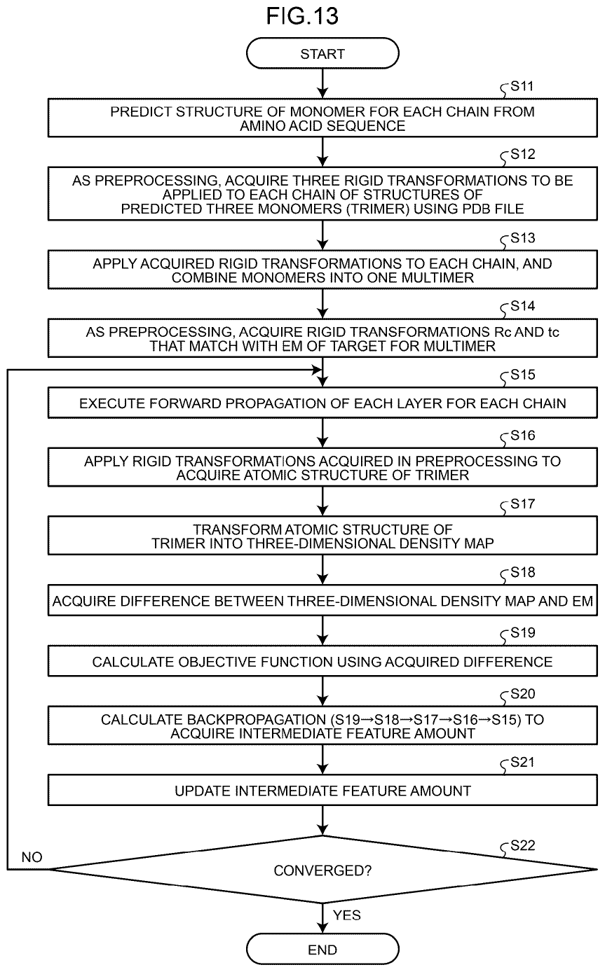

Here, a flowchart of the predictive control process that is executed by the information processing device 1 will be described with reference to FIG. 13. FIG. 13 is a diagram illustrating an example of the flowchart of the predictive control process according to the first embodiment.

As illustrated in FIG. 13, the information processing device 1 predicts a structure of a monomer for each chain from an amino acid sequence (Step S11). For example, the structure prediction model MO11 predicts an atomic structure of a monomer forming protein from the amino acid sequence for each chain.

As preprocessing, the information processing device 1 acquires three rigid transformations that match with each chain of atomic structures of predicted three monomers (trimer) using the PDB file 21 (Step S12). For example, the preprocessing unit 12 executes the first preprocessing. The information processing device 1 applies the acquired rigid transformations to each chain, and combines the three monomers into one multimer (Step S13).

As preprocessing, the information processing device 1 acquires the rigid transformations Rc and tc that match with the EM (three-dimensional density map) of the target for the multimer (Step S14). For example, the preprocessing unit 12 executes the second preprocessing.

The information processing device 1 executes forward propagation of each layer for each chain (Step S15). For example, the information processing device 1 executes Expression (6). Here, the information processing device 1 does not update the intermediate feature amount rci.

The information processing device 1 applies the rigid transformations acquired in the preprocessing of S12 and S14 to acquire an atomic structure of the trimer (Step S16). For example, the information processing device 1 executes Expression (5).

The information processing device 1 transforms the atomic structure of the trimer into the three-dimensional density map (Step S17). For example, the information processing device 1 executes Expression (7), Expression (8), and Expression (9).

The information processing device 1 acquires a difference between the transformed three-dimensional density map and the EM (three-dimensional density map) of the target (Step S18). For example, the information processing device 1 executes Expression (10).

The information processing device 1 calculates an objective function using the acquired difference (Step S19). For example, the information processing device 1 executes Expression (11).

The information processing device 1 calculates backpropagation (S19→S18→S17→S16→S15) to acquire an intermediate feature amount (Step S20). For example, by executing Expression (12) to Expression (16), the information processing device 1 calculates backpropagation to acquire an intermediate feature amount. The information processing device 1 updates the acquired intermediate feature amount to the structure prediction model MO11 (Step S21).

The information processing device 1 determines whether the structure prediction by the structure prediction model MO11 is converged (Step S22). When the information processing device 1 determines that the structure prediction is not converged (Step S22; No), the information processing device 1 transitions to Step S15 to execute the next structure prediction.

On the other hand, when the information processing device 1 determines that the structure prediction is converged (Step S22; Yes), the information processing device 1 ends the predictive control process.

As described above, the information processing device 1 according to the first embodiment updates the intermediate feature amount of the structure prediction model MO11 to reduce a difference between the three-dimensional density map that is transformed from the predicted structure output as the prediction result from the structure prediction model MO11 using the interpolation expression satisfying the density conservation law and a three-dimensional density map that is different from the three-dimensional density map. Accordingly, the information processing device 1 according to the first embodiment can predict various atomic structures. The information processing device 1 can predict various atomic structures in a realistic time by updating the intermediate feature amount of the structure prediction model MO11 to reduce the difference between the three-dimensional density map transformed from the predicted structure and the correct three-dimensional density map.

### Variations of First Embodiment

Incidentally, in the description of the information processing device 1 according to the first embodiment, the transformation unit 13 transforms the atomic structure to be output as the prediction result from the structure prediction model MO11 into a three-dimensional density map (EM) for limiting the atomic structure by using the interpolation expression satisfying the density conservation law as the differentiable form. However, the transformation unit 13 is not limited to the interpolation expression satisfying the density conservation law, and may transform the atomic structure into the three-dimensional density map (EM) for limiting the atomic structure, for example, using an approximate expression by a spherically symmetric Gaussian distribution.

When the limiting information for limiting the atomic structure is a molecular shape, a molecular electron density map (three-dimensional density map) can be used. By using an atom position and an atomic scattering factor given, the molecular three-dimensional density map can be accurately calculated based on physics. However, the three-dimensional density map acquired from the atomic scattering factor has many undulations. Therefore, when a search is executed by using the three-dimensional density map as the limiting information as it is, the undulations may interrupt the search. For example, the search may remain in a local solution.

Accordingly, by approximating a functional form of the atomic scattering factor and smoothing a three-dimensional density map transformed from the approximated functional form in advance, smoothness during a search can be improved while maintaining accuracy of the molecular shape.

The atomic scattering factor f(q) is expressed by four Gaussian functions and a constant term as represented by Expression (19). The atomic scattering factor f(q) can be approximated to be expressed by four or less Gaussian functions as represented by Expression (20). When N represents each of 1, 2, 3, and 4, the approximation is executed. a, b, and c are fitting parameters, and fitting may be newly executed.

$$f ( q ) = {\sum }_{i = 1}^{4} {a}_{i} exp ( - {{b}_{i} ( \frac{q}{4 π} )}^{2} ) + c \qquad Expression ( 19 )$$

$$f ( q ) = {\sum }_{i = 1}^{{N}_{f}} {a}_{i} exp ( - {{b}_{i} ( \frac{q}{4 π} )}^{2} ) \qquad Expression ( 20 )$$

In addition, as an example of a method of smoothing (obtaining a smooth image of) the three-dimensional density map, a low-pass filter (a cutoff wave number is f_{c}) in a wave number space can be used. In a case where the low-pass filter in the wave number space is used, when a target resolution (the degree of smoothing) is represented by R(A) and a molecular size parameter is represented by L(A), the number N of voxels per dimension can be represented by Expression (21). The molecular size parameter L can be accurately predicted by using the accurately calculated three-dimensional density map and a molecular surface defined from the three-dimensional density map.

$$N = 2 L / R = L {f}_{c} \qquad Expression ( 21 )$$

This way, using Expression (20) and Expression (21), the transformation unit 13 transforms the atomic structure of the trimer of the prediction result into the three-dimensional density map for limiting the atomic structure. Expression (20) is a transformation function from the atomic structure into the three-dimensional density map for limiting the atomic structure, and is a differentiable transformation function.

As a result, the information processing device 1 according to the present variation updates the intermediate feature amount of the structure prediction model MO11 to reduce a difference between the three-dimensional density map that is transformed from the predicted structure output as the prediction result from the structure prediction model MO11 using the approximate expression by the spherically symmetric Gaussian distribution and a three-dimensional density map that is different from the three-dimensional density map. Accordingly, the information processing device 1 according to the present variation can predict various atomic structures. The information processing device 1 can predict various atomic structures in a realistic time by updating the intermediate feature amount of the structure prediction model MO11 to reduce the difference between the three-dimensional density map transformed from the predicted structure and the correct three-dimensional density map.

### 4. Configuration of Information Processing Device

### Second Embodiment

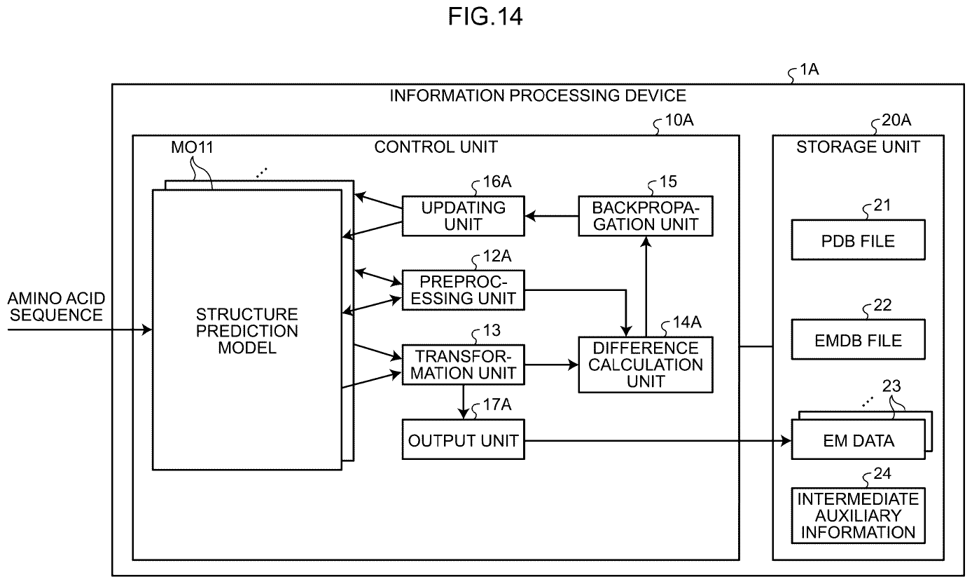

FIG. 14 is a diagram illustrating an example of a functional configuration of an information processing device according to the second embodiment. The information processing device 1A illustrated in FIG. 14 is an example of a computer that executes the predictive control process. As illustrated in FIG. 14, the information processing device 1A includes a control unit 10A and a storage unit 20A. The same description as that of the first embodiment will be appropriately omitted.

The storage unit 20A includes the PDB file 21, the EMDB file 22, the EM data 23, and intermediate auxiliary information 24. The descriptions of the PDB file 21, the EMDB file 22, and the EM data 23 are the same as that of the first embodiment, and thus will be omitted.

The intermediate auxiliary information 24 is intermediate auxiliary information that is generated by the predictive control process. The intermediate auxiliary information 24 may be intermediate auxiliary information that is collectively generated based on the starting point structure, or may be intermediate auxiliary information that is dynamically generated in the process of stepwise fitting. The intermediate auxiliary information 24 is stored in the storage unit 20A by an output unit 17A.

The control unit 10A includes the plurality of structure prediction models MO11, a preprocessing unit 12A, the transformation unit 13, a difference calculation unit 14A, the backpropagation unit 15, an updating unit 16A, and the output unit 17A. The descriptions of the transformation unit 13 and the backpropagation unit 15 are the same as that of the first embodiment, and thus will be omitted.

For example, in addition to the process of the preprocessing unit 12, the preprocessing unit 12A generates the intermediate auxiliary information. For example, based on the auxiliary information E′ₜ (corresponding to the first information) corresponding to the predicted structure Sₜ by the structure prediction model MO11 and the target auxiliary information E_{T} (corresponding to the second information) that is the fitting target thereof, the preprocessing unit 12A generates intermediate auxiliary information Eₜ₊₁ to the intermediate auxiliary information E_{T-1} (corresponding to the intermediate information) as the intermediate information between the auxiliary information E′ₜ and the target auxiliary information E_{T}. The preprocessing unit 12A generates the intermediate auxiliary information Eₜ₊₁ to the intermediate auxiliary information E_{T-1}, for example, using the predicted structure Sₜ at the present time as a starting point behavior. When the predicted structure Sₜ is the predicted structure of the initial prediction (that is, the predicted structure S₀), the preprocessing unit 12A generates the intermediate auxiliary information E₁ to the intermediate auxiliary information E_{T-1}.

The preprocessing unit 12A acquires, for example, the first information and the second information that is a fitting target of the first information. For example, the preprocessing unit 12A acquires the first information that is the three-dimensional density map transformed from the predicted structure Sₜ and the second information that is the three-dimensional density map that is input by a user as a correct three-dimensional density map together with an amino acid sequence.

The preprocessing unit 12A may collectively generate or may dynamically generate, for example, the intermediate auxiliary information Eₜ₊₁ to the intermediate auxiliary information E_{T-1}. During the collective generation, the intermediate auxiliary information Eₜ₊₁ to the intermediate auxiliary information E_{T-1} are initially determined. On the other hand, during the dynamic generation, the intermediate auxiliary information Eₜ₊₁ is initially determined, the intermediate auxiliary information Eₜ₊₂ is determined after the initial fitting, and the intermediate auxiliary information Eₜ₊₃ is determined after the second fitting. By repeating this process T times, the intermediate auxiliary information Eₜ₊₁ to the intermediate auxiliary information E_{T-1} are determined.

The preprocessing unit 12A generates the intermediate auxiliary information Eₜ₊₁ to the intermediate auxiliary information E_{T-1}, for example, such that the index d(Eₜ, E_{T}) satisfies Expression (2). The preprocessing unit 12A generates the intermediate auxiliary information Eₜ₊₁ to the intermediate auxiliary information E_{T-1}, for example, such that the next intermediate auxiliary information to be used for fitting is in a state of being likely to reach the final target auxiliary information E_{T}.

The preprocessing unit 12A collectively generates the intermediate auxiliary information Eₜ₊₁ to the intermediate auxiliary information E_{T-1}, for example, using a method that is based on linear interpolation between density maps or a method that is based on density interpolation based on optimal transport. In addition, the preprocessing unit 12A dynamically generates the intermediate auxiliary information Eₜ₊₁ to the intermediate auxiliary information E_{T-1}, for example, using a method that is based on linear interpolation between the auxiliary information E′ₜ₋₁ and the target auxiliary information E_{T} or a method of generating a plurality of intermediate auxiliary information candidates as the intermediate auxiliary information around the auxiliary information E′ₜ₋₁ and setting, as the intermediate auxiliary information Eₜ, the intermediate auxiliary information candidate for minimizing the index d(Eₜ, E_{T}).

The preprocessing unit 12A collectively generates one or more three-dimensional density maps as the intermediate auxiliary information, for example, based on linear interpolation between three-dimensional density maps of the first information corresponding to the predicted structure Sₜ at the present time (corresponding to the predicted structure S₀ of the initial prediction) and the second information. The preprocessing unit 12A collectively generates one or more three-dimensional density maps as the intermediate auxiliary information, for example, based on density interpolation between three-dimensional density maps based on optimal transport of the first information corresponding to the predicted structure Sₜ at the present time (corresponding to the predicted structure S₀ of the initial prediction) and the second information.

The preprocessing unit 12A dynamically generates one or more three-dimensional density maps as the intermediate auxiliary information, for example, in the process of stepwise fitting based on linear interpolation between three-dimensional density maps of the first information corresponding to the predicted structure Sₜ at the present time (corresponding to the predicted structure S₀ of the initial prediction) and the second information. The preprocessing unit 12A dynamically generates one or more three-dimensional density maps as the intermediate auxiliary information, for example, in the process of generating a plurality of candidates of the intermediate information around the first information corresponding to the predicted structure Sₜ at the present time (corresponding to the predicted structure S₀ of the initial prediction) and fitting the first information to the candidates stepwise while setting, as the intermediate auxiliary information, the candidate for minimizing the index d(Eₜ, E_{T}) representing ease of reaching the second information.

The preprocessing unit 12A generates the intermediate auxiliary information, for example, such that the index d(Eₜ, E_{T}) representing ease of reaching the second information satisfies a predetermined condition. The preprocessing unit 12A generates the intermediate auxiliary information, for example, based on the index d(Eₜ, E_{T}) that is based on a cross-correlation between three-dimensional density maps. The preprocessing unit 12A generates the intermediate auxiliary information, for example, based on the index d(Eₜ, E_{T}) based on a Wasserstein distance. The preprocessing unit 12A generates the intermediate auxiliary information, for example, based on the index d(Eₜ, E_{T}) that is based on an inter-structure distance based on a structure probability estimated by a structure probability distribution estimation method in a three-dimensional density map space. The preprocessing unit 12A generates the intermediate auxiliary information, for example, under the predetermined condition that the next intermediate auxiliary information to be used for fitting is likely to reach the second information.

The difference calculation unit 14A executes, for example, the same process as that of the difference calculation unit 14. However, unlike the difference calculation unit 14, the difference calculation unit 14A calculates a difference between the three-dimensional density map corresponding to the atomic structure of the trimer of the prediction result and the three-dimensional density map of the intermediate structure instead of the difference between the three-dimensional density map corresponding to the atomic structure of the trimer of the prediction result and the three-dimensional density map of the target in the EMDB file 22. That is, in the second embodiment, unlike the first embodiment, the three-dimensional density map that is target of fitting is the intermediate auxiliary information instead of the target auxiliary information E_{T}. When the predicted structure Sₜ is the predicted structure of the initial prediction (that is, the predicted structure S₀), the difference calculation unit 14A calculates a difference between the auxiliary information E′₀ and the intermediate auxiliary information E₁. When the predicted structure Sₜ is the predicted structure S₁, the difference calculation unit 14A calculates a difference between the auxiliary information E′₁ and the intermediate auxiliary information E₂.

For example, in addition to the process of the updating unit 16, the updating unit 16A updates the intermediate feature amount of the structure prediction model MO11 through the processes of the difference calculation unit 14A and the backpropagation unit 15 based on the intermediate auxiliary information Eₜ₊₁ to the intermediate auxiliary information E_{T-1} that is generated by the preprocessing unit 12A. The updating unit 16A updates the intermediate feature amount, for example, the first intermediate feature amount RE1 or the second intermediate feature amount RE2. When the predicted structure Sₜ is the predicted structure of the initial prediction (that is, the predicted structure S₀), the updating unit 16A updates the intermediate feature amount H₀ based on the auxiliary information E′₀ and the intermediate auxiliary information E₁. When the predicted structure Sₜ is the predicted structure S₁, the updating unit 16A updates the intermediate feature amount H₁ based on the auxiliary information E′₁ and the intermediate auxiliary information E₂. The updating unit 16A updates the intermediate feature amount, for example, such that a difference between the next intermediate auxiliary information to be used for fitting and the first information is minimized by backpropagation based on the difference.

For example, when fitting is executed using the target auxiliary information E_{T} as a target, the updating unit 16A calculates the cross-correlation C1 between the auxiliary information E′ₜ corresponding to the predicted structure Sₜ at the present time and the target auxiliary information E_{T} for each repetition of a predetermined number of times (m times). For example, when the calculated cross-correlation C1 exceeds a predetermined threshold TH1, or when the repetition of the predetermined number of times (n times) ends, the updating unit 16A stores the updated intermediate feature amount Hₜ and the auxiliary information E′ₜ corresponding to the predicted structure Sₜ, and sequentially executes fitting after changing t to t+1.

For example, in addition to the process of the output unit 17, the output unit 17A stores the three-dimensional density map transformed by the transformation unit 13 in the storage unit 20A as the EM data 23.

### Flowchart of Predictive Control Process

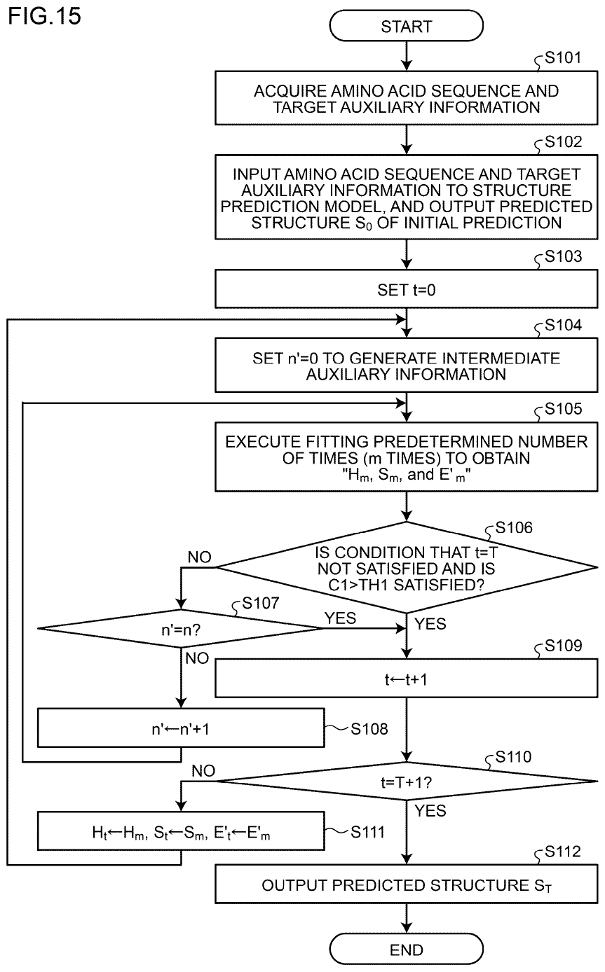

Here, a flowchart of the predictive control process that is executed by the information processing device 1A will be described with reference to FIG. 15. FIG. 15 is a diagram illustrating an example of the flowchart of the predictive control process according to the second embodiment.

As illustrated in FIG. 15, the information processing device 1A acquires the amino acid sequence IN1 and the target auxiliary information E_{T} (Step S101).

The information processing device 1A inputs the acquired amino acid sequence IN1 and the acquired target auxiliary information E_{T} to the structure prediction model MO11, and outputs the predicted structure S₀ of the initial prediction (Step S102).

The information processing device 1A sets t=0 (Step S103).

The information processing device 1A sets the number n′ of updates of the intermediate feature amount to n′=0 to generate the intermediate auxiliary information Eₜ₊₁ to the intermediate auxiliary information E_{T-1} (Step S104).

The information processing device 1A executes fitting a predetermined number of times (m times) to obtain an intermediate feature amount Hₘ, a predicted structure Sₘ, and auxiliary information E′ₘ (Step S105).

The information processing device 1A determines whether a condition that t=T is not satisfied and the cross-correlation C1 exceeds the threshold TH1 is satisfied (Step S106).

When Step S106 is No, the information processing device 1A determines whether n′ reaches a predetermined number of times (n times) (n′=n?) (Step S107).

Further, when Step S107 is No, the information processing device 1A substitutes n′+1 for n′ (n′←n′+1) (Step S108), and returns to Step S105.

On the other hand, when Step S106 is Yes, or when Step S107 is Yes, the information processing device 1A substitutes t+1 for t (t←t+1) (Step S109).

The information processing device 1A determines whether t reaches T+1 (t=T+1?) (Step S110).

When Step S110 is No, the information processing device 1A substitutes Hₘ for Hₜ (Hₜ←Hₘ), substitutes Sₘ for Sₜ (Sₜ←Sₘ), and substitutes E′ₘ for E′ₜ (E′ₜ←E′ₘ) (Step S111), and returns to Step S104.

On the other hand, when Step S110 is Yes, the information processing device 1A outputs the predicted structure S_{T} (Step S112). This way, the information processing device 1A outputs the predicted structure S_{T} based on the intermediate feature amount that is updated a predetermined number of times as the target structure corresponding to the input amino acid sequence.

Hereinabove, the embodiments regarding the device disclosed herein have been described. However, the present invention may be implemented in various different forms, in addition to the above-described embodiments. Accordingly, hereinafter, other embodiments of the present invention will be described.

Information including the process procedure, the control procedure, the specific names, and various data and parameters described in the documents and illustrated in the drawings of the first embodiment and the second embodiment may be freely changed unless otherwise specified.

In addition, specific forms of distributing or integrating components of each device are not limited to the drawings. That is, all or some of the components may be functionally or physically distributed or integrated in any units depending on various loads, usage conditions, and the like. Further, all or some of process functions of each device may be implemented by a CPU and a program that is analyzed and executed by the CPU, or may be implemented as hardware by wired logic.

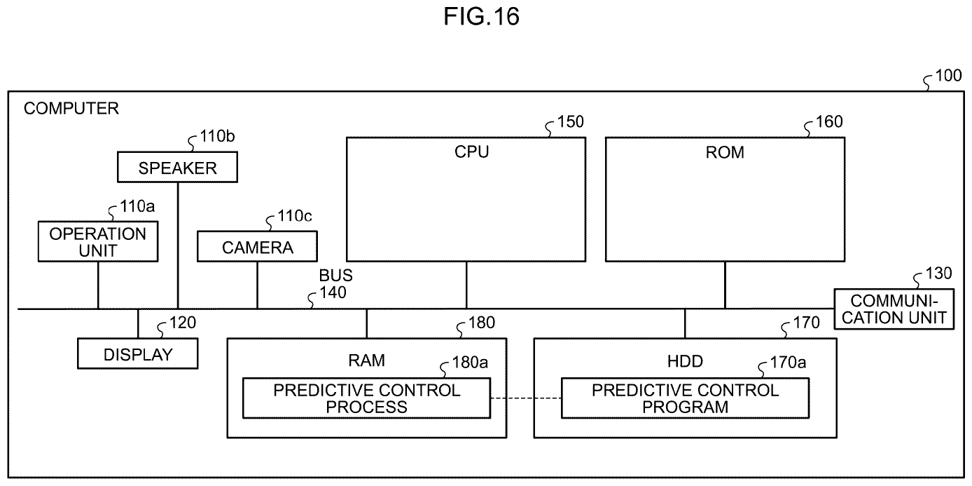

Various processes described in the first embodiment and the second embodiment can be implemented by a computer such as a personal computer or a workstation executing a program that is prepared in advance. Accordingly, hereinafter, an example of the computer that executes a control program having the same functions as that of first embodiment and the second embodiment will be described using FIG. 16.

FIG. 16 is a diagram illustrating a hardware configuration example. As illustrated in FIG. 16, a computer 100 includes an operation unit 110a, a speaker 110b, a camera 110c, a display 120, and a communication unit 130. Further, this computer 100 includes a CPU 150, a ROM 160, an HDD 170, and a RAM 180. These units 110 to 180 are connected via a bus 140.

As illustrated in FIG. 16, the HDD 170 stores a predictive control program 170a that exhibits the same functions as the structure prediction model MO11, the preprocessing unit 12, the preprocessing unit 12A, the transformation unit 13, the difference calculation unit 14, the difference calculation unit 14A, the backpropagation unit 15, the updating unit 16, the updating unit 16A, the output unit 17, and the output unit 17A described in the first embodiment and the second embodiment. The predictive control program 170a may be distributed or integrated as in each of the components including the structure prediction model MO11, the preprocessing unit 12, the preprocessing unit 12A, the transformation unit 13, the difference calculation unit 14, the difference calculation unit 14A, the backpropagation unit 15, the updating unit 16, the updating unit 16A, the output unit 17, and the output unit 17A illustrated in FIGS. 9 and 14. That is, the HDD 170 does not need to store all pieces of the data described in the first embodiment and the second embodiment, and only needs to store the data to be used for the process.

In this environment, the CPU 150 reads the predictive control program 170a from the HDD 170, and loads the predictive control program 170a to the RAM 180. As a result, the predictive control program 170a functions as a predictive control process 180a as illustrated in FIG. 16. In the predictive control process 180a, various types of data read from the HDD 170 are loaded to an area allocated to the predictive control process 180a in a storage area in the RAM 180, and various processes are executed using the loaded various types of data. Examples of the process that is executed by the predictive control process 180a may include the processes illustrated in FIGS. 13 and 15. In the CPU 150, all the processing units described in the first embodiment and the second embodiment do not need to be operated, or a processing unit corresponding to a process as an execution target may be virtually implemented.

The predictive control program 170a does not need to be stored in the HDD 170 or the ROM 160 from the beginning. For example, the predictive control program 170a may be stored in a “portable physical medium”, for example, a flexible disk, a so-called FD, a CD-ROM, a DVD disk, a magneto-optic disk, or an IC card to be inserted into the computer 100. The computer 100 may acquire the predictive control program 170a from the portable physical medium to execute the predictive control program 170a. In addition, the predictive control program 170a may be stored in advance in another computer or a server device connected to the computer 100 via a public network, the Internet, a LAN, a WAN, or the like. The predictive control program 170a that is stored as described above may be downloaded to the computer 100 to be executed.

Regarding the above-described embodiments, the following supplementary notes will be further disclosed.
(1)

A predictive control method comprising:
acquiring first information that corresponds to a predicted structure predicted by a structure prediction model based on machine learning, and second information that is a fitting target of the first information;
generating intermediate information between the first information and the second information based on the acquired first information and the acquired second information; and
updating an intermediate feature amount of the structure prediction model based on the generated intermediate information, by a processor.
(2)

The predictive control method according to (1),
wherein the first information is a three-dimensional density map transformed from the predicted structure, and
the second information is a correct three-dimensional density map input from a user.
(3)

The predictive control method according to (1) or (2),
wherein the structure prediction model outputs the predicted structure based on the intermediate feature amount that is updated a predetermined number of times as a target structure corresponding to an input amino acid sequence.
(4)

The predictive control method according to any one of (1) to (3),
wherein, in the updating process, the intermediate feature amount is updated to minimize a difference between the next intermediate information to be used for fitting and the first information by backpropagation based on the difference.
(5)

The predictive control method according to any one of (1) to (4),
wherein, in the generating process, the intermediate information is generated such that an index representing ease of reaching the second information satisfies a predetermined condition.
(6)

The predictive control method according to (5),
wherein, in the generating process, the intermediate information is generated under the predetermined condition that the next intermediate information to be used for fitting is likely to reach the second information.
(7)

The predictive control method according to (5) or (6),
wherein, in the generating process, the intermediate information is generated based on the index that is based on a cross-correlation between three-dimensional density maps, the index that is based on a Wasserstein distance, or the index that is based on an inter-structure distance based on a structure probability estimated by a structure probability distribution estimation method in a three-dimensional density map space.
(8)

The predictive control method according to any one of (1) to (7),
wherein, in the generating process, one or more three-dimensional density maps are collectively generated as the intermediate information based on linear interpolation between three-dimensional density maps of the first information corresponding to the predicted structure of initial prediction and the second information, or density interpolation between three-dimensional density maps based on optimal transport of the first information corresponding to the predicted structure of initial prediction and the second information.
(9)

The predictive control method according to any one of (1) to (7),
wherein, in the generating process, one or more three-dimensional density maps are dynamically generated as the intermediate information based on linear interpolation between three-dimensional density maps of the first information corresponding to the predicted structure at a present time and the second information, or in the process of generating a plurality of candidates of the intermediate information around the first information corresponding to the predicted structure at the present time and fitting the first information to the candidates stepwise while setting, as the intermediate information, the candidate for minimizing an index representing ease of reaching the second information.
(10)

The predictive control method according to any one of (1) to (9),
wherein the structure prediction model is a model that outputs the predicted structure as 3D structure data of an organic compound when an amino acid sequence is input.
(11)

The predictive control method according to any one of (1) to (10),
wherein the structure prediction model includes a plurality of layers, and generates an intermediate feature amount of each of the plurality of layers.
(12)

An information processing device comprising:
a processor configured to:
acquire first information that corresponds to a predicted structure predicted by a structure prediction model based on machine learning, and second information that is a fitting target of the first information;
generate intermediate information between the first information and the second information based on the acquired first information and the acquired second information; and
update an intermediate feature amount of the structure prediction model based on the generated intermediate information.
(13)

A non-transitory computer-readable recording medium having stored therein a program that causes a computer to execute a process comprising:
acquiring first information that corresponds to a predicted structure predicted by a structure prediction model based on machine learning, and second information that is a fitting target of the first information;
generating intermediate information between the first information and the second information based on the acquired first information and the acquired second information; and
updating an intermediate feature amount of the structure prediction model based on the generated intermediate information.

According to one embodiment, an effect capable of improving accuracy of fitting during prediction of a target structure is exhibited.

All examples and conditional language recited herein are intended for pedagogical purposes of aiding the reader in understanding the invention and the concepts contributed by the inventors to further the art, and are not to be construed as limitations to such specifically recited examples and conditions, nor does the organization of such examples in the specification relate to a showing of the superiority and inferiority of the invention. Although the embodiments of the present invention have been described in detail, it should be understood that the various changes, substitutions, and alterations could be made hereto without departing from the spirit and scope of the invention.

What is claimed is:

1. A predictive control method comprising:
acquiring first information that corresponds to a predicted structure predicted by a structure prediction model based on machine learning, and second information that is a fitting target of the first information;
generating intermediate information between the first information and the second information based on the acquired first information and the acquired second information; and
updating an intermediate feature amount of the structure prediction model based on the generated intermediate information, by a processor.

2. The predictive control method according to claim 1, wherein the first information is a three-dimensional density map transformed from the predicted structure, and
the second information is a correct three-dimensional density map input from a user.

3. The predictive control method according to claim 1, wherein the structure prediction model outputs the predicted structure based on the intermediate feature amount that is updated a predetermined number of times as a target structure corresponding to an input amino acid sequence.

4. The predictive control method according to claim 1, wherein the intermediate feature amount is updated to minimize a difference between the next intermediate information to be used for fitting and the first information by backpropagation based on the difference.

5. The predictive control method according to claim 1, wherein the intermediate information is generated such that an index representing ease of reaching the second information satisfies a predetermined condition.

6. The predictive control method according to claim 5, wherein the intermediate information is generated under the predetermined condition that the next intermediate information to be used for fitting is likely to reach the second information.

7. The predictive control method according to claim 5, wherein the intermediate information is generated based on the index that is based on a cross-correlation between three-dimensional density maps, the index that is based on a Wasserstein distance, or the index that is based on an inter-structure distance based on a structure probability estimated by a structure probability distribution estimation method in a three-dimensional density map space.

8. The predictive control method according to claim 1, wherein one or more three-dimensional density maps are collectively generated as the intermediate information based on linear interpolation between three-dimensional density maps of the first information corresponding to the predicted structure of initial prediction and the second information, or density interpolation between three-dimensional density maps based on optimal transport of the first information corresponding to the predicted structure of initial prediction and the second information.

9. The predictive control method according to claim 1, wherein one or more three-dimensional density maps are dynamically generated as the intermediate information based on linear interpolation between three-dimensional density maps of the first information corresponding to the predicted structure at a present time and the second information, or in the process of generating a plurality of candidates of the intermediate information around the first information corresponding to the predicted structure at the present time and fitting the first information to the candidates stepwise while setting, as the intermediate information, the candidate for minimizing an index representing ease of reaching the second information.

10. An information processing device comprising:
a processor configured to:
acquire first information that corresponds to a predicted structure predicted by a structure prediction model based on machine learning, and second information that is a fitting target of the first information;
generate intermediate information between the first information and the second information based on the acquired first information and the acquired second information; and
update an intermediate feature amount of the structure prediction model based on the generated intermediate information.

11. A non-transitory computer-readable recording medium having stored therein a program that causes a computer to execute a process comprising:
acquiring first information that corresponds to a predicted structure predicted by a structure prediction model based on machine learning, and second information that is a fitting target of the first information;
generating intermediate information between the first information and the second information based on the acquired first information and the acquired second information; and
updating an intermediate feature amount of the structure prediction model based on the generated intermediate information.
